(19) United States
(12) Patent Application Publication
(10) Pub. No.: US 2025/0259743 A1
(43) Pub. Date: Aug. 14, 2025

(54) HEALTH KIOSK SYSTEM AND METHOD FOR MANAGING HEALTH OF USERS

(71) Applicant: Phigital Care Private Limited, Bengaluru (IN)

(72) Inventor: Satyender Goel, Karnal (IN)

(21) Appl. No.: 19/053,646

(22) Filed: Feb. 14, 2025

(30) Foreign Application Priority Data
Feb. 14, 2024 (IN) 202441010012

(51) Int. Cl. G16H 40/63; G16H 10/20; G16H 50/70

(52) U.S. Cl. CPC G16H 40/63; G16H 10/20; G16H 50/70

(57) ABSTRACT

The present invention discloses a health kiosk system and method for managing health of users. The health kiosk system is configured to manage the health of one or more users based on a dynamic extraction of one or more parameters. The health kiosk system comprises a health kiosk unit, and a plurality of subsystems. The health kiosk unit comprises one or more embedded sensors, a plurality of electrocardiogram (ECG) leads, and one or more server units. The plurality of subsystems provides personalised insights, facilitates disease prevention, and supports wellness programs. The health kiosk system is configured to allow healthcare professionals to trigger additional tests remotely for remote health assessments. The health kiosk system obtain consent data from the one or more users to recommend optimised preferences for telemedicine, research, and analysis.

### CROSS-REFERENCES TO RELATED APPLICATIONS

This application claims priority to Indian Patent Application bearing No. 202441010012 filed on Feb. 14, 2024, entitled, “HEALTH KIOSK SYSTEM AND METHOD FOR MANAGING HEALTH OF USERS”, the disclosure of which is incorporated herein by reference in its entirety for all purposes.

### FIELD OF INVENTION

Embodiments of the present invention relate to healthcare technology and more particularly relate toa health kiosk system for managing health of one or more users based on a dynamic extraction of one or more parameters.

### BACKGROUND

Telemedicine is a transformative approach to healthcare delivery, employing telecommunications technology to connect healthcare professionals with one or more users remotely. This innovative practice enables virtual consultations, diagnosis, and treatment, fostering accessibility to medical expertise irrespective of geographical distances. Through video calls, secure messaging, and digital tools, the telemedicine enhances convenience for the one or more users and reduces barriers to healthcare access. The telemedicine plays a crucial role in preventive care, chronic disease management, and timely medical interventions, thereby revolutionising a traditional healthcare model for a more interconnected and user-centric future.

However, traditional telemedicine methods face challenges including limited accessibility for the one or more users without adequate technology and internet access, potential security and privacy concerns associated with digital platforms, and a lack of comprehensive integration with existing healthcare systems. The reliance on technology may also pose obstacles for older populations and those not comfortable with the digital tools. In the context of cardiac health, electrocardiogram (ECG) readings are pivotal for accurate diagnostics, especially in conditions requiring detailed insights into cardiac electrical activity. The conventional methods of acquiring ECG data in virtual consultations rely on the one or more user's self-placement of the ECG devices, which may lead to inaccuracies and hinder the effectiveness of remote cardiac assessments.

In an existing technology, a system and method for intelligent patient interface exam station is disclosed. The system provides the one or more users with diagnostic tools, audio guidance, and video guidance to perform clinical-grade diagnostic measurements of key vital signs reliably and accurately. This is accomplished by using an automated remote end-to-end medical diagnostic system that monitors equipment usage for accuracy. The diagnostic system analyses responses, measurement data, and user-related information to generate diagnostic data that may be shared with the healthcare professionals. However, the system lacks the capability for the healthcare professionals to initiate remote activation of one or more medical screening tests.

Similarly in another existing technology, a medical kiosk system and method are disclosed. The system comprises a plurality of sensors and devices. The system is configured to communicate medical information to a server and interact with the healthcare professionals. The server is configured to communicate the medical information of the one or more users to the system. The system is configured to provide the one or more users with a prescription and a medication. However, the system may lack the ability to conduct a comprehensive and personalised assessment of the health of the one or more users. The automated nature of the medical kiosk system may not fully replace the nuanced evaluation that the healthcare professional may provide during a face-to-face consultation. Personalised healthcare requires a thorough understanding of a medical history, lifestyle, and circumstances of the one or more users, which is challenging for the medical kiosk system to assess accurately.

There are various technical problems with the health kiosk system in the prior art. In the existing technology, Vulnerabilities in the health kiosk system's security may expose the sensitive diagnostic data to unauthorised access. Difficulty in integrating the diagnostic data from the health kiosk system into existing electronic health records (EHR) and healthcare databases may impede the seamless flow of information within the healthcare system. Inadequate data storage capacity and issues with data retrieval may impact the ability of the health kiosk system to store and access historical health information. Moreover, ensuring active and dynamic one or more user consent in real-time for data collection during remote health interactions remains a vital yet challenging aspect. The traditional health kiosks often lack the sophistication required for detailed cardiac assessments. Further, the health kiosk system faces difficulty in obtaining real-time and comprehensive health data, which is critical for accurate diagnostics and personalised healthcare. Existing virtual healthcare systems lack integrated solutions for performing detailed health screenings and diagnostics remotely.

Therefore, there is a need for a system to address the aforementioned issues by managing the health of the one or more users efficiently, providing personalised healthcare insights, and facilitating proactive health management. There is also the need for the system to ensure accessible and real-time healthcare, thereby promoting early intervention and empowering the one or more users in their well-being.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In order to overcome the above deficiencies of the prior art, the present disclosure is to solve the technical problem by providing a health kiosk system for managing health of one or more users based on dynamic extraction of one or more parameters.

In accordance with an embodiment of the present invention, the health kiosk system for managing the health of the one or more users based on the dynamic extraction of the one or more parameters is provided. The system comprises a health kiosk unit, one or more hardware processors, a data repository unit, and a plurality of subsystems. The health kiosk unit comprises one or more embedded sensors, a plurality of electrocardiogram (ECG) leads, and one or more server units. The plurality of subsystems comprise a decision-making subsystem, a survey generation subsystem, a user interaction subsystem, a data processing subsystem, a remote assistant subsystem, and a remote test triggering subsystem.

In an embodiment, the one or more embedded sensors are configured to generate sensor data. The plurality of ECG leads are operatively positioned at diverse places in the health kiosk unit. The plurality of ECG leads are configured to generate cardiac electrical activity data of the one or more users associated with a user profile. The one or more server units are operatively connected to the health kiosk unit via a communication network. The one or more embedded sensors comprise at least one of blood pressure sensors, body composition analysis sensors, temperature sensors, and oxygen saturation sensors. The plurality of ECG leads is one of: three leads, six leads, and twelve leads operatively integrated into the health kiosk unit at the diverse places. The six leads of the plurality of ECG leads are positioned at diverse places comprise a left-palm placement, right-palm placement, left-hand wrist placement, right-hand wrist placement, left ankles placement, and right ankles placement.

In an embodiment, the one or more hardware processors are operatively connected to the one or more server units. The data repository unit is operatively coupled to the one or more hardware processors. The data repository unit comprises the plurality of subsystems in form of programmable instructions executable by the one or more hardware processors.

In an embodiment, the decision-making subsystem is configured with computer-implemented intelligent models to determine a type of data to be collected from the one or more users based on the user profile. The computer-implemented intelligent models comprise at least one of a: self-aware artificial intelligence model, large language model, supervised artificial intelligence and machine learning model, unsupervised artificial intelligence and machine learning model, deep learning model, simple and multimodal analytics including at least one of: regression models, Gaussian Mixture Models (GMMs), and K-Nearest Neighbours Algorithm models.

In an embodiment, the survey generation subsystem is configured to generate one or more queries for obtaining first data based on the determined type of data to be collected from the one or more users. The survey generation subsystem is configured to dynamically generate health-related queries based on the user's historical health data and ongoing health conditions. The first data comprises at least one of: biographical information, health history data, user preferences, survey responses, choice selections for the consent data, and medical records.

In an embodiment, the user interaction subsystem is configured to receive the first data from the one or more users associated with the user profile through a user interface for initiating personalised health-related interactions within the health kiosk system.

In an embodiment, the data processing subsystem is configured to process at least one of the: sensor data, cardiac electrical activity data, and first data for determining the one or more parameters of the one or more users associated with the user profile. The one or more parameters comprises at least one of a: blood pressure, body composition, body temperature, ECG data, weight and height measurements, optical acuities data, electroencephalogram, Lung function test, urine test, oxygen saturation, Body Mass Index (BMI), blood glucose, and lipid profile.

In an embodiment, the remote assistant subsystem is configured to connect the one or more users with a healthcare professional based on the one or more parameters for virtual assistance. The remote assistant subsystem is configured with at least one of: audio conferencing capabilities and video conferencing capabilities for activating at least one of a: camera, and microphone embedded in the health kiosk unit.

In an embodiment, the remote test triggering subsystem is configured to allow the healthcare professional to trigger a medical screening test on the one or more users interacted with the health kiosk unit for managing the health of the one or more users based on dynamic extraction of the one or more parameters.

In an embodiment, the plurality of subsystems comprises a consent data obtaining subsystem. The consent data obtaining subsystem is configured to obtain consent data from the one or more users to recommend optimised preferences. The consent data enable the one or more users to manage and optimise data-sharing preferences for at least one of: telemedicine, research, and analysis.

In accordance with an embodiment of the present disclosure, a method for managing the health of the one or more users based on the dynamic extraction of the one or more parameters is provided. In a first step, the method includes, generating, by the one or more embedded sensors, the sensor data. In the next step, the method includes, generating, by the plurality of ECG leads, the cardiac electrical activity data of the one or more users associated with the user profile.

In the next step, the method includes, determining, by the decision-making subsystem configured with the computer-implemented intelligent models, the type of data to be collected from the one or more users based on the user profile. In the next step, the method includes, generating, by the survey generation subsystem, the one or more queries for obtaining the first data based on the determined type of data to be collected from the one or more users.

In the next step, the method includes, receiving, by the user interaction subsystem, the first data from the one or more users associated with the user profile through the user interface to initiate the personalised health-related interactions within the health kiosk system. In the next step, the method includes, processing, by the data processing subsystem, at least one of the: sensor data, cardiac electrical activity data, and the first data for determining the one or more parameters of the one or more users associated with the user profile.

In the next step, the method includes, connecting, by the remote assistant subsystem, the one or more users with the healthcare professional based on the one or more parameters for the virtual assistance. In the next step, the method includes, allowing, by the remote test triggering subsystem, the healthcare professional to trigger the medical screening test on the one or more users interacted with the health kiosk unit for managing the health of the one or more users based on the dynamic extraction of the one or more parameters.

In the next step, the method includes, obtaining, by the consent data obtaining subsystem, the consent data from the one or more users to recommend the optimised preferences. The consent data enable the one or more users to manage and optimise data-sharing preferences for at least one of the: telemedicine, research, and analysis.

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The disclosure will be described and explained with additional specificity and detail with the accompanying figures in which:

FIG. 1 illustrates an exemplary block diagram representation of a network architecture of a health kiosk system for managing health of one or more users based on a dynamic extraction of one or more parameters, in accordance with an embodiment of the present disclosure;

FIG. 2 illustrates an exemplary block diagram representation of the health kiosk system as shown in FIG. 1 for managing the health of the one or more users based on the dynamic extraction of the one or more parameters, in accordance with an embodiment of the present disclosure;

FIG. 3 illustrates an exemplary flow chart depicting a method managing the health of the one or more users based on the dynamic extraction of the one or more parameters, in accordance with an embodiment of the present disclosure;

FIG. 4A illustrates an exemplary real image of a health kiosk unit, in accordance with an embodiment of the present invention; and

FIG. 4B illustrates an exemplary flow diagram depicting the health kiosk system for managing the health of the one or more users based on the dynamic extraction of the one or more parameters, in accordance with an embodiment of the present invention.

Further, those skilled in the art will appreciate that elements in the figures are illustrated for simplicity and may not have necessarily been drawn to scale. Furthermore, in terms of the method steps, chemical compounds, equipment and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE PRESENT INVENTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present invention relate toa health kiosk system for managing health of one or more users based on dynamic extraction of one or more parameters.

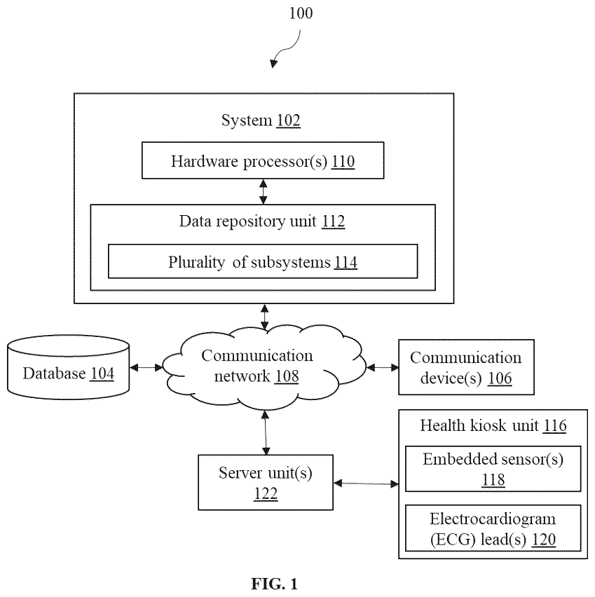

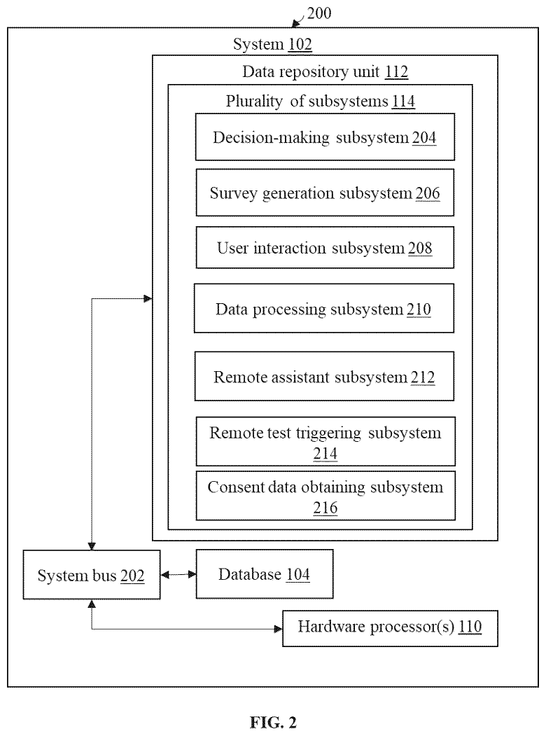

FIG. 1 refers to an exemplary block diagram representation of a network architecture 100 of the health kiosk system 102 for managing the health of the one or more users based on the dynamic extraction of the one or more parameters, in accordance with an embodiment of the present disclosure.

According to an exemplary embodiment of the present disclosure, FIG. 1 depicts the network architecture 100 may include the health kiosk system 102 (hereinafter referred to as the system 102), a database 104, and one or more communication devices 106. The system 102 comprises a health kiosk unit 116, one or more hardware processors 110, a data repository unit 112, and a plurality of subsystems 114. The health kiosk unit 116 comprises one or more embedded sensors 118, a plurality of electrocardiogram (ECG) leads 120, and one or more server units 122. The system 102 may be communicatively coupled to the database 104 via a communication network 108. The communication network 108 may be a wired communication network and/or a wireless communication network. The database 104 may include, but not limited to, storing, and managing data related to the one or more parameters and user profiles. The database 104 may be any kind of database such as, but not limited to, relational databases, non-relational databases, document databases, dedicated databases, dynamic databases, monetised databases, scalable databases, cloud databases, distributed databases, any other databases, and a combination thereof. The database 104 is configured to support the functionality of the system 102 and enables efficient data retrieval and storage for various aspects associated with the one or more parameters, first data, and consent data. The one or more communication devices 106 may be digital devices, computing devices and/or networks. The one or more communication devices 106 may include, but not limited to, a mobile device, a smartphone, a Personal Digital Assistant (PDA), a tablet computer, a phablet computer, a wearable computing device, a laptop, a desktop, and the like. In an alternative embodiment, the one or more communication devices 106 are configured to establish digital communication with the health kiosk unit 116. The one or more users operate the one or more communication devices 106 to transmit their medical history data securely to the health kiosk unit 116. This upload functionality involves the exchange of digital information between the one or more communication devices 106 and the health kiosk unit 116, ensuring the seamless transfer of comprehensive medical history data. The one or more communication devices 106 serve as user-friendly interfaces, allowing the one or more users to share their health information conveniently and contribute to a centralized repository accessible by the health kiosk unit 116 for enhanced healthcare assessments and consultations.

This integrated network architecture 100 facilitates seamless communication and data exchange, enabling the system 102 to operate cohesively for managing the health of the one or more users based on the dynamic extraction of the one or more parameters. The system 102 capability to monitor the health of the one or more users is underpinned by the effective collaboration among the system 102, the database 104, and the one or more communication devices 106 within the communication network 108.

Further, the system 102 may be implemented by way of a single device or a combination of multiple devices that may be operatively connected or networked together. The system 102 may be implemented in hardware or a suitable combination of hardware and software. The system 102 may be a hardware device including the one or more hardware processors 110 executing machine-readable program instructions for managing the health of the one or more users based on the dynamic extraction of the one or more parameters. Execution of the machine-readable program instructions by the one or more hardware processors 110 may enable the system 102 to dynamically recommend course of action sequences for managing the health of the one or more users based on the dynamic extraction of the one or more parameters. The course of action sequences may involve various steps or decisions taken for decision-making, survey generating, user interacting, data processing, and remote assistance. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The one or more server units 122 may be optimal-performance computing units that are operatively connected to the health kiosk unit 116 via the communication network 108. The one or more server units 122a redesigned to handle complex computations and efficiently process large datasets. The one or more server units 122 are configured to orchestrate various subsystems, execute algorithms, and manage the overall functionality of the system 102 for the dynamic extraction of the one or more parameters. The one or more server units 122 are configured as a computational backbone, processing user queries, extracting the one or more parameters, and connecting the one or more users with a healthcare professional.

The health kiosk unit 116 incorporates the one or more embedded sensors 118 configured to generate sensor data. The plurality of ECG leads 120 are placed at diverse locations within the health kiosk unit 116. The plurality of ECG leads 120 are precisely configured to capture and generate comprehensive cardiac electrical activity data. The cardiac electrical activity data of the one or more users is associated with the user profiles, forming a vital component in the dynamic extraction of the one or more parameters. By strategically employing the plurality of ECG leads 120, the health kiosk unit 116 ensures accurate and detailed monitoring of the cardiac activity of the one or more users, contributing to a holistic approach for managing the health of the one or more users.

The one or more embedded sensors 118 may include, but not limited to, at least one of: blood pressure sensors, body composition analysis sensors, temperature sensors, oxygen saturation sensors, and the like. The plurality of ECG leads 120 is one of: three leads, six leads, twelve leads, and the like operatively integrated into the health kiosk unit 116 at the diverse places. The six leads of the plurality of ECG leads 120 are positioned at diverse places may include, but not limited to a left-palm placement, right-palm placement, left-hand wrist placement, right-hand wrist placement, left ankles placement, right ankles placement, and the like.

The one or more hardware processors 110 may include, for example, microprocessors, microcomputers, microcontrollers, digital signal processors, central processing units, state machines, logic circuits, and/or any devices that manipulate data or signals based on operational instructions. Among other capabilities, the one or more hardware processors 110 may fetch and execute computer-readable instructions in the data repository unit 112 operationally coupled with the system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, ports, routers, repeaters, firewall devices, network devices, databases, network attached storage devices, servers, assets, machinery, instruments, facility equipment, emergency management devices, image capturing devices, any other devices, and combination thereof. A person skilled in the art should not be limiting the components/subsystems shown in FIG. 1.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognise that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices that were known in the art.

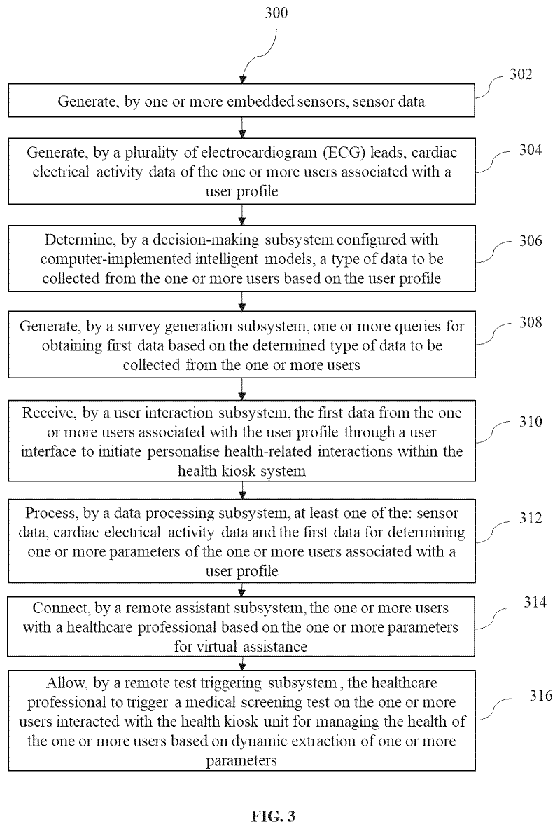

FIG. 2 refers to an exemplary block diagram representation 200 of the system as shown in FIG. 1 for managing the health of the one or more users based on the dynamic extraction of the one or more parameters, in accordance with an embodiment of the present disclosure.

In an exemplary embodiment, the system 102 comprises the one or more hardware processors 110, the data repository unit 112, and the database 104. The one or more hardware processors 110, the data repository unit 112, and the database 104 are communicatively coupled through a system bus 202 or any similar mechanism. The data repository unit 112 is operatively coupled to the one or more hardware processors 110. The data repository unit 112 comprises the plurality of subsystems 114 in the form of programmable instructions executable by the one or more hardware processors 110.

In an exemplary embodiment, the plurality of subsystems 114 comprise a decision-making subsystem 204, a survey generation subsystem 206, a user interaction subsystem 208, a data processing subsystem 210, a remote assistant subsystem 212, and a remote test triggering subsystem 214. The plurality of subsystems 114 manages the health of the one or more users based on the dynamic extraction of the one or more parameters. The division of tasks among the plurality of subsystems 114 assists in achieving a modular and efficient approach to the overall functionality of the system 102.

The one or more hardware processors 110, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The data repository unit 112 may be a non-transitory volatile memory and a non-volatile memory. The data repository unit 112 may be coupled to communicate with the one or more hardware processors 110, such as being a computer-readable storage medium. The one or more hardware processors 110 may execute machine-readable instructions and/or source code stored in the data repository unit 112. A variety of machine-readable instructions may be stored in and accessed from the data repository unit 112. The data repository unit 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the data repository unit 112 includes the plurality of subsystems 114 stored in the form of machine-readable instructions on any of the above-mentioned storage media and may be in communication with and executed by the one or more hardware processors 110.

In an exemplary embodiment, the decision-making subsystem 204 is intricately designed with computer-implemented intelligent models that enable the decision-making subsystem 204 to discern a type of data to be gathered from the one or more users. By dynamically adapting the data collection strategy, the decision-making subsystem 204 ensures a personalised and efficient approach to health monitoring, contributing to more targeted and effective healthcare interventions. The computer-implemented intelligent models may include, but not limited to, at least one of a: self-aware artificial intelligence model, large language model, supervised artificial intelligence and machine learning model, unsupervised artificial intelligence and machine learning model, deep learning model, simple and multimodal analytics including at least one of: regression models, Gaussian Mixture Models (GMMs), K-Nearest Neighbours Algorithm models and the like.

In an exemplary embodiment, the self-aware artificial intelligence model endows the system 102 with self-awareness, enabling the system 102 to adapt and evolve in response to changing the user profiles and system dynamics, enhancing the overall responsiveness of the platform. The large language model leveraging extensive language models, the system 102 is configured to interpret and process user inputs with linguistic nuance, facilitating a more natural and context-aware interaction during consultations and data collection. The supervised artificial intelligence and machine learning model is configured to employ labelled training data, this labelled training data enhances decision-making by learning patterns from past interactions, enabling the system 102 to make informed predictions and recommendations tailored to the user profiles.

The unsupervised artificial intelligence and machine learning model explores and identifies inherent patterns within user data without explicit guidance, uncovering hidden insights and potential correlations that contribute to a more comprehensive understanding of user health profiles. The deep learning model integrates deep neural networks to excel in recognizing intricate patterns within complex health data, allowing for more accurate diagnostics and personalized health insights during virtual consultations. The simple and multimodal analytics utilises a combination of regression models for predicting trends, GMMs for clustering and density estimation, and KNN algorithm models for proximity-based decision-making, this simple and multimodal analytics ensures a diverse and thorough analysis of user health data, supporting a multifaceted understanding of individual health profiles.

In an exemplary embodiment, the survey generation subsystem 206 is designed to dynamically create one or more queries for obtaining the first data in alignment with the determined type of data to be collected from the one or more users. The survey generation subsystem 206 tailors the one or more queries based on the individual user's historical health data and ongoing health conditions. The first data may include, but not limited to, at least one of: biographical information, health history data, user preferences, survey responses, choice selections for the consent data, medical records, and the like. This personalised approach ensures that the collected data is comprehensive, relevant, and reflective of the one or more user's health, thereby contributing to a more nuanced and insightful health assessment within the system 102.

In an exemplary embodiment, the user interaction subsystem 208 is configured to facilitate the reception of the first data from the one or more users, linked to the respective user profiles of the one or more users, employing a user interface within the system 102. The user interaction subsystem 208 serves as a gateway for personalised health-related interactions within the system 102, fostering the one or more users' engagement and enabling the initiation of the tailored health-related interactions based on the received first data. By seamlessly integrating the first data, the system 102 ensures a user-centric approach, enhancing the overall effectiveness of health-related interactions and interventions within the system 102. The user profiles are created by providing at least one of a: an identity card (ID), a unique number issued by a government, the unique number by a private entity, biometrics, and the like.

In an exemplary embodiment, the data processing subsystem 210 is adeptly configured to handle and analyse at least one of the: sensor data, cardiac electrical activity data and first data for determining the one or more parameters of the one or more users associated with the user profile. The one or more parameters may include, but not limited to, at least one of a: blood pressure, body composition, body temperature, ECG data, weight and height measurements, optical acuities data, electroencephalogram, Lung function test, urine test, oxygen saturation, Body Mass Index (BMI), blood glucose, lipid profile, and the like. The data processing subsystem 210 enables a thorough assessment of the one or more users' health conditions, contributing to the nuanced and the holistic understanding within the system 102.

In an exemplary embodiment, the remote assistant subsystem 212 is configured to establish a connection between the one or more users and the healthcare professional via the communication network 108, leveraging the determined one or more parameters for virtual assistance. The remote assistant subsystem 212 is configured with at least one of: audio conferencing capabilities and video conferencing capabilities for activating at least one of a: camera, and microphone embedded in the health kiosk unit 116. Through these functionalities, the one or more users may engage in real-time communication with the healthcare professional, thereby facilitating remote consultations and support. The remote assistant subsystem 212 enhances the accessibility of healthcare services by providing a platform for personalised health related interactions, allowing the healthcare professional to remotely assess the one or more users' health conditions, provide guidance, and make informed recommendations, thereby ensuring timely and responsive virtual assistance within the system 102.

In an exemplary embodiment, the remote test triggering subsystem 214 is designed to empower the healthcare professional to initiate a medical screening test for the one or more users interacting with the health kiosk unit 116, thereby contributing to the comprehensive management of the health of the one or more users based on the dynamically extracted one or more parameters. In another exemplary embodiment, for initiating the medical screening test, the healthcare professional generates a request for specific medical screening tests through the user interface. The request includes details including the type of test, duration, and any specific instructions for system 102 to execute the medical screening tests. The request generated by the healthcare professional is securely transmitted to the health kiosk unit 116 in real-time. This communication may involve encrypted messages to maintain data security and integrity during transmission. Upon receiving the request, the health kiosk unit 116 notifies the one or more users about the proposed additional medical screening tests. The system 102 may be configured with information about the medical screening tests, their purpose, and any potential risks. The one or more users are prompted to provide explicit consent for the medical screening tests. Upon receiving the user consent, the health kiosk unit 116 executes the requested medical screening tests. This execution may involve activating specific sensors, diagnostic tools, and integrated functionalities to perform the required assessments. As the additional medical screening tests are conducted, the results are generated in real-time. The system 102 securely transmits the results back to the healthcare professional's interface, enabling immediate access and analysis.

By facilitating the remote initiation of the medical screening test, the remote test triggering subsystem 214 enhances the adaptability of the system 102, thereby allowing the healthcare professional to further assess specific health indicators and make informed decisions to optimise the overall health management of the one or more users involved in the system 102.

In an exemplary embodiment, the plurality of subsystems 114 further comprise a consent data obtaining subsystem 216. The consent data obtaining subsystem 216 is tailored to obtain the consent data from the one or more users, facilitating the recommendation of optimised preferences. The consent data is obtained through at least one of: the microphones, a secondary display unit, a physical keyboard on the health kiosk unit 116, and the like. The consent data obtaining subsystem 216 is configured to gather the consent data from the one or more users, empowering the one or more users to manage and optimise their data-sharing preferences for at least one of: telemedicine, research, analysis, and the like. By obtaining the consent data, the system 102 ensures transparency and respects privacy choices of the one or more users, fostering a personalised and user-centric approach to the employment of the consent data.

FIG. 3 refers to an exemplary flow chart depicting a method 300 for managing the health of the one or more users based on the dynamic extraction of the one or more parameters, in accordance with an embodiment of the present disclosure.

According to an exemplary embodiment of the present disclosure, the method 300 for managing the health of the one or more users based on the dynamic extraction of the one or more parameters is disclosed. At step 302, the method 300 includes the one or more embedded sensors configured to generate the sensor data, thereby capturing a diverse range of health-related information. Subsequently, at step 304, the method 300 includes the plurality of ECG leads, takes centre stage in generating the cardiac electrical activity data specific to the one or more users associated with the user profiles.

At step 306, the method 300 includes the decision-making subsystem, which is equipped with the computer-implemented intelligent models. The decision-making subsystem dynamically determines the type of data to be collected from the one or more users based on the user profile, showcasing a personalised approach to the health data gathering. At step 308, the method 300 includes the survey generation subsystem, generating the one or more queries to obtain the first data. The one or more queries are based on the determined type of data collected from the one or more users. This cohesive process ensures a targeted and the user-centric approach to the first data collection within the system.

At step 310, the method 300 includes the user interaction subsystem, receiving the first data from the one or more users through the user interface, establishing a vital link to initiate the personalised health-related interactions within the system. At step 312, the method 300 includes the data processing subsystem, processing at least one of the: sensor data, cardiac electrical activity data, and the first data. The processing is aimed at determining the one or more parameters of the one or more users associated with the user profile, thus laying the foundation for the nuanced and thorough assessment of the health of the one or more users.

At step 314, the method 300 includes the remote assistant subsystem, connecting the one or more users with the healthcare professional, leveraging the dynamically extracted one or more parameters for the virtual assistance. This step facilitates real-time interactions, allowing the healthcare professional to provide guidance and support. At step 316, the method 300 includes the remote test triggering subsystem, granting the healthcare professional the capability to initiate the medical screening test for the one or more users engaged with the health kiosk unit.

Furthermore, the method 300 includes the consent data obtaining subsystem, which is responsible for gathering the consent data from the one or more users. The consent data obtaining subsystem allows the one or more users to articulate and specify their preferences, optimising their control over the consent data sharing. The consent data enables the one or more users to manage and optimise data-sharing preferences for at least one of the: telemedicine, research, analysis, and the like.

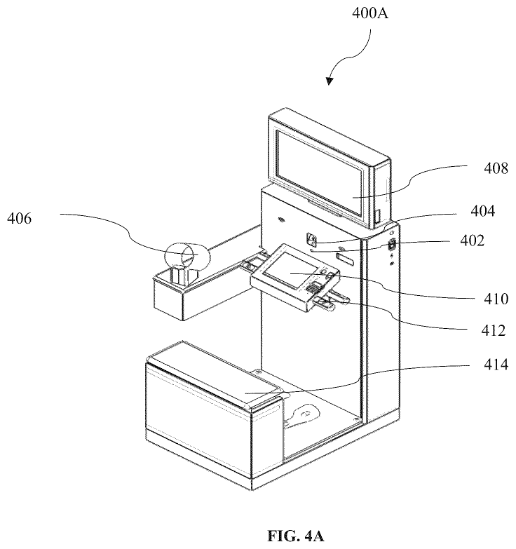

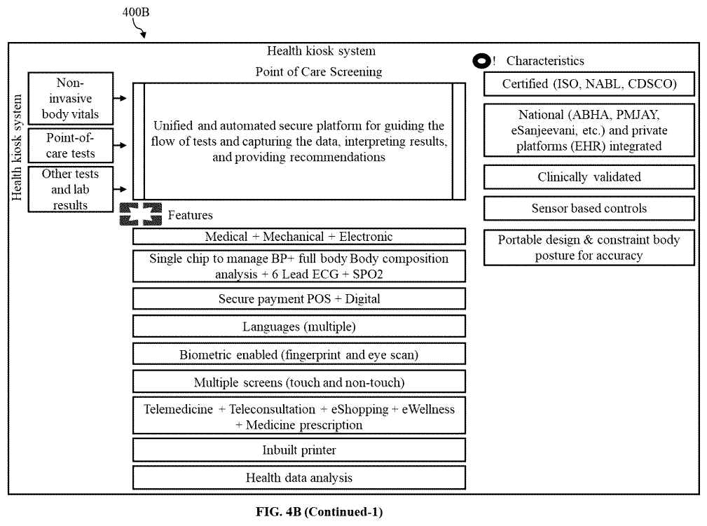

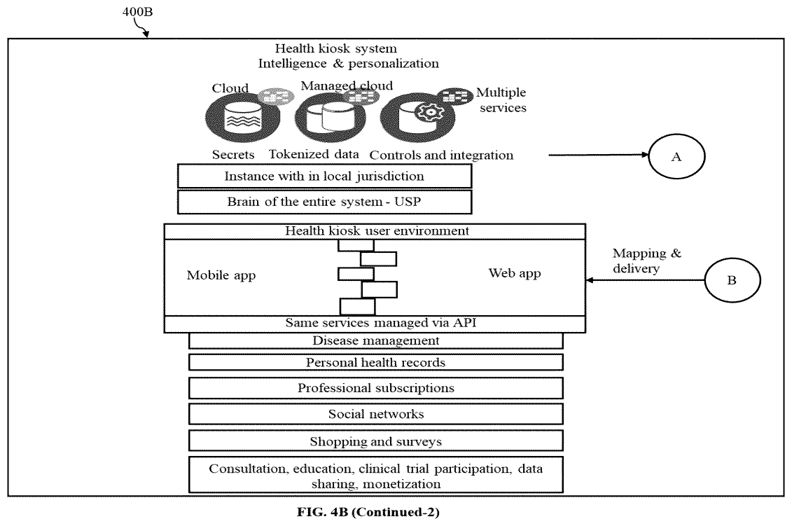

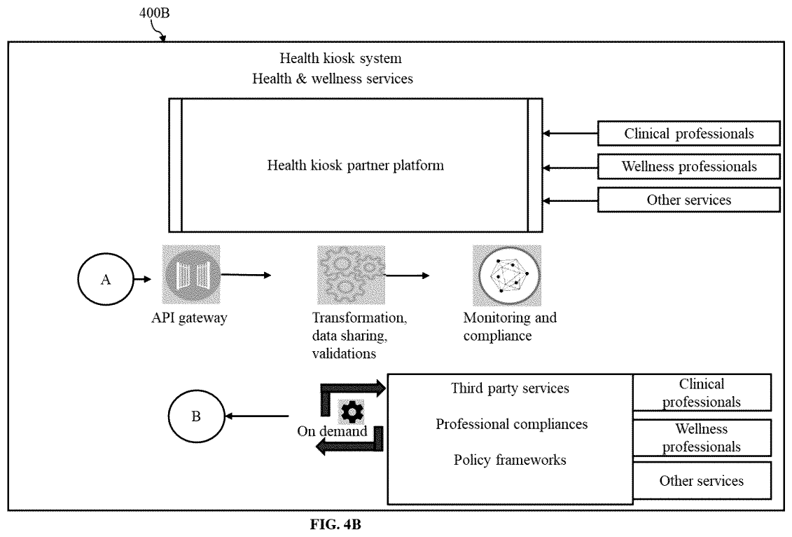

FIG. 4A refers to an exemplary real image 400A of the health kiosk unit 116, in accordance with an embodiment of the present invention; and

FIG. 4B refers to an exemplary flow diagram 400B depicting the system for managing the health of the one or more users based on the dynamic extraction of the one or more parameters, in accordance with an embodiment of the present invention.

In an exemplary embodiment, the health kiosk unit 116 is intricately designed with distinct components to serve diverse functionalities. The primary display unit 408, tailored for advertising purposes, is precisely calibrated at, but not limited to, 19 inches, providing a vibrant and expansive canvas for promotional content. Complementing this, the secondary display unit 410 is configured at, but not limited to, 10.4 inches, catering to user interactions with an interface optimized for clarity and ease of use. In terms of power dynamics, the health kiosk unit 116 is engineered with a power consumption specification of, but not limited to, 160 watts per hour, striking a balance between operational efficiency and energy conservation. The robust power supply for the health kiosk unit 116 operates seamlessly within the range of 180V to 230V, ensuring adaptability to diverse power infrastructures. The physical dimensions of the health kiosk unit 116 are meticulously crafted to meet specific spatial requirements. With a height of 1696 mm, a length of 796.4 mm, and a width of 835 mm, the unit is designed to optimize both vertical and horizontal spatial considerations. Moreover, the weight of the health kiosk unit 116 is documented at 100 kg to 150 kg, reflecting a balance between structural integrity and practical manoeuvrability.

The camera 402, and the microphone 404 are embedded in the health kiosk unit 116. The camera 402, and the microphone 404 are configured to connect the one or more users and the healthcare professionals via the communication network 108 for the virtual assistance. The health kiosk unit 116 is configured with an adjustable seat 414 to accommodate physically challenged one or more users and the one or more users of varying heights. This configuration ensures that the plurality of ECG leads 120 may be appropriately positioned in the health kiosk unit 116 comes in contact with the one or more users for the effective cardiac electrical activity data collection. In an exemplary embodiment, the adjustable seat 414 not only enhances accessibility but also promotes a user-friendly experience, allowing the one or more users with diverse physical abilities to comfortably engage with the system 102. This thoughtful design underscores the commitment to providing equitable healthcare solutions and ensures that the health kiosk unit 116 is adaptable to the unique needs of a broad user demographic, enhancing the accuracy and efficiency of the sensor-based data gathering process.

Further, the health kiosk unit 116 is configured with a blood pressure monitor port 406. The blood pressure monitor port 406 in the health kiosk unit 116 functions as a specialized interface for measuring the blood pressure of the one or more users. The blood pressure monitor port 406 is equipped with the blood pressure sensors. The one or more users conveniently insert their hand into the blood pressure monitor port 406, allowing for the secure attachment of the blood pressure sensors. The health kiosk unit 116 is configured with a holder 412. The holder 412 is equipped with the one or more embedded sensors 118. The holder 412 is configured to measure heartbeats of the one or more users.

The system 102 integrates physical and digital elements to revolutionise the healthcare management. Operating on a unified and automated secure platform, the system 102 guides the seamless flow of tests for determining the one or more parameters and captures the health-related information from non-invasive body vitals, point-of-care tests, and various lab results. The system 102 is managed by a single chip and handles the blood pressure analysis, full-body composition analysis, the six leads of the plurality of ECG leads 120, and peripheral capillary oxygen saturation (SPO2), forming the brain of the system 102 with clinical validation.

The system 102 caters to clinical and wellness professionals, third-party services, and complies with professional standards and policy frameworks. The system 102 encompasses features including at least one of: telemedicine, teleconsultation, e-shopping, e-wellness, and medicine prescription, ensuring the comprehensive health and wellness experience. The system 102 further enhances disease management, personal health records, professional subscriptions, social networks, shopping, surveys, and various health services. The system 102 not only transforms the healthcare landscape but also provides a dynamic and integrated approach to personalised well-being.

The system 102 is accessible via at least one of a: mobile application and web application and harmonizes various services through application programming interface (API) management. With an inbuilt printer, the system 102 truly represents a comprehensive and transformative paradigm in the health and wellness, employing advanced technologies to integrate the diverse healthcare facets for the improved accessibility, efficiency, and user experience.

Built on a foundation of cutting-edge technologies, the system 102 boasts characteristics including secure cloud management, tokenised data protection, compliance monitoring, and the like. The adaptability and scalability of the system 102 are highlighted by its ability to integrate with various services, ensuring a versatile and the user-centric healthcare experience. Furthermore, a localised instance of the system 102 within specific jurisdictions emphasises a commitment to regulatory compliance and data governance, reinforcing its role as an innovative and secure solution in the evolving landscape of the digital health and wellness.

The system 102 provides a multifaceted approach encompassing consultation, education, clinical trial participation, data sharing, and monetisation. The system 102 facilitates the education by providing valuable information to the one or more users, empowering the one or more users with knowledge for informed decision-making. Additionally, the system 102 enables the clinical trial participation, contributing to medical research advancements. Emphasising the data sharing, the system 102 prioritises user privacy while promoting collaborative efforts for broader healthcare insights. The monetisation aspects involve sustainable business models to support the system's 102 continuous improvement and expansion, ensuring ongoing benefits for the one or more users managing the health proactively.

The system 102 integrates cutting-edge technology to ensure secure payment processing, featuring a robust point of sale (POS) coupled with digital language options for enhanced accessibility. With support for multiple languages, the one or more users may interact with the system 102 in their preferred language, fostering inclusivity. The implementation of biometric authentication, employing fingerprint and eye scan technology, enhances security and the one or more users' identification, providing the seamless and the personalised health management experience. The system 102 is equipped with multiple screens, accommodating both touch interactions and non-touch interactions, thereby providing the versatile interface for the one or more users.

The system 102 exemplifies a commitment to quality and compliance by incorporating certifications including an International Organisation for Standardization (ISO), a National Accreditation Board for Testing and Calibration Laboratories (NABL), and a Central Drugs Standard Control Organisation (CDSCO), ensuring adherence to international standards and regulatory requirements. The system 102 seamlessly integrates with national platforms including Ayushman Bharat Health Account (ABHA), Pradhan Mantri Jan Arogya Yojana (PMJAY), and eSanjeevani, fostering interoperability and extending its reach across diverse healthcare ecosystems. The system 102 caters to both public healthcare domains and private healthcare domains by supporting Electronic Health Records (EHR) on private platforms. Employing the plurality of sensor-based controls, the system 102 guarantees the accuracy of the one or more parameters extraction, providing the one or more users with the reliable and the real-time health insights. The portable design coupled with constraint body posture considerations enhances the one or more users' comfort and engagement, making the system 102 the holistic and user-friendly solution for managing the health of the one or more users.

Numerous advantages of the present disclosure may be apparent from the discussion above. In accordance with the present disclosure, the system for managing the health of the one or more users based on the dynamic extraction of the one or more parameters is provided. The system is streamlined health screening with an incorporated registration system, ready for an Accredited Social Health Activist (ASHA) and an Auxiliary Nurse Midwife (ANM) deployment, and the seamless payment collection. This alternative not only enhances cost savings but also optimises the workflow by combining registration and financial transactions for services including medicine. The system is designed to cater to the one or more users while they are on the move. This solution ensures continuous health support for the one or more users, addressing the healthcare needs regardless of their location. The system leveraging the existing healthcare professionals in a public sector to provide the virtual assistance during their non-busy hours. An integrated platform that combines the benefits of Ayushman Bharat Digital Mission (ABDM) led programs including the ABHA ID, an Unified Health Interface (UHI), and a Health Claims Exchange (HCX) protocol, thereby providing a one-stop solution for all the health-related needs.

An expedited alternative for non-invasive body vital screenings, providing over 22 essential parameters valued at approximately [symbol] 1000 to [symbol] 2000, all achievable within approximately 3 minutes. The system ensures the efficiency and the accessibility in both clinic and remote settings without the need for direct supervision. A comprehensive alternative integrating blood and urine screening, encompassing an additional more than 25 parameters. Managed reports are efficiently delivered on at least one of the: mobile application and web application associated with the one or more communication devices, providing the one or more users with detailed insights into the healthcare insights. The system provides daily health tips and guided training on how to employ the system effectively. An inclusive alternative facilitating teleconsultations with in-house doctors. The system features language-based selection options, ensuring accessibility for diverse populations and enabling the one or more users to communicate comfortably with the healthcare professionals. The system provides the comprehensive virtual assistance alternative with a unified statewide branding approach. This solution ensures consistency in the healthcare delivery, establishing a trusted and recognisable brand for the virtual assistance across a state.

The intelligent system is configured to automatically detect certain demographics and features of the one or more users including height, gender, and the like. Further, the system also measures ambient data points including humidity, weather, temperature, pollution, and the like. The system collects geographical data, other social determinants like education, employment, and political views. The system may be incorporated in public places, railway stations, bus stops, airports, parks, government buildings, Resident Welfare Associations (RWAs), office buildings, retail stores, banks, standalone sheds, covered area similar to a phone booth, hospitals, clinic, pharmacy, lab centre, and any physical location with the area to hold the health kiosk unit. The one or more users may log in into the system by employing a username, a password, the biometrics, a quick response (QR) code, the ID, the unique number, single sign-on authenticated by a third-party provider, and the like. The health-related information is conveyed to the one or more users and the system in any format via paper printouts, emails, the mobile application, web browser, and the like. The one or more users may provide feedback and updated information to the system.

The system is configured to provide swift deployment and scalability, covering 1000 locations within a three-month timeframe. This approach ensures the seamless rollout while maintaining the ability to scale quickly and efficiently. The system provides excellent expansion options with a focus on low-cost, high-impact visibility. This approach maximises reach while optimising the costs, providing a scalable model that emphasises effectiveness and efficiency. An alternative launch strategy for the state, leveraging a compelling tagline such as ‘Health On-the-Go’ or ‘Empowering Health Everywhere.’ This approach aims to resonate with the target audience and promote the new campaign effectively. An alternative management approach where the application is fully managed by IHL (India Health Link), with a strategic partnership to scale the impact. The system ensures efficient operations and impactful scaling. The system incorporates real-time analytics to dynamically select high-value services over time. This data-driven approach allows for continuous refinement of offerings based on usage patterns and their impact, ensuring a responsive and evolving healthcare platform.

The order functionality in the system is configured to streamline the management of the health of the one or more users by providing various services. The one or more users may effortlessly place the orders for a range of vital health-related elements including appointments, deliveries, and interactions, covering a broad spectrum of items including medicines, medical devices, food, beverages, and supplements. The system supports diverse transaction types, allowing the one or more users the flexibility to engage in voluntary, paid, credit-based, and rewarded transactions, catering to various financial preferences. Additionally, the orders may be conveniently placed using coupons, codes, and the IDs. The system provides the real-time analytics to provide a clear and dynamic representation of a Return on Investment (ROI) at a statewide level. The system provides insightful comparison charts showcasing the prevalence of the diseases, enabling stakeholders to make informed decisions based on current health trends. In addition to the disease analytics, the system incorporates wellness programs, specifically tailored for heart health management, obesity management, and diabetes management. The health kiosk unit is configurable to an adjustable seat for physically challenged one or more users. The health kiosk unit is constructed from a at least one of: metal or alternative strong material with at least one of a: powder coat and metallic paint finish.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

I/We claim:

1. A health kiosk system for managing health of one or more users based on dynamic extraction of one or more parameters, comprising:
a health kiosk unit comprises:
one or more embedded sensors configured to generate sensor data;
a plurality of electrocardiogram (ECG) leads operatively positioned at diverse places in the health kiosk unit, configured to generate cardiac electrical activity data of the one or more users associated with a user profile;
one or more server units operatively connected to the health kiosk unit via a communication network;
one or more hardware processors operatively connected to the one or more server units;
a data repository unit operatively coupled to the one or more hardware processors, wherein the data repository unit comprises a plurality of subsystems in form of programmable instructions executable by the one or more hardware processors, wherein the plurality of subsystems comprise:
a decision-making subsystem configured with computer-implemented intelligent models to determine a type of data to be collected from the one or more users based on the user profile;
a survey generation subsystem configured to generate one or more queries for obtaining first data based on the determined type of data to be collected from the one or more users;
a user interaction subsystem configured to receive the first data from the one or more users associated with the user profile through a user interface for initiating personalised health-related interactions within the health kiosk system;
a data processing subsystem configured to process at least one of the: sensor data, cardiac electrical activity data and the first data for determining the one or more parameters of the one or more users associated with the user profile;
a remote assistant subsystem configured to connect the one or more users with a healthcare professional based on the one or more parameters for virtual assistance; and
a remote test triggering subsystem configured to allow the healthcare professional to trigger a medical screening test on the one or more users interacted with the health kiosk unit for managing the health of the one or more users based on dynamic extraction of the one or more parameters.

2. The health kiosk system as claimed in claim 1, wherein the plurality of subsystems comprise a consent data obtaining subsystem,
the consent data obtaining subsystem configured to obtain consent data from the one or more users to recommend optimised preferences,
the consent data enable the one or more users to manage and optimise data-sharing preferences for at least one of: telemedicine, research, and analysis.

3. The health kiosk system as claimed in claim 1, wherein the one or more embedded sensors comprise at least one of blood pressure sensors, body composition analysis sensors, temperature sensors, and oxygen saturation sensors.

4. The health kiosk system as claimed in claim 1, wherein the plurality of electrocardiogram (ECG) leads is one of: three leads, six leads, and twelve leads operatively integrated into the health kiosk unit at the diverse places,

5. The health kiosk system as claimed in claim 4, wherein the six leads of the plurality of electrocardiogram (ECG) leads positioned at diverse places comprise a left-palm placement, right-palm placement, left-hand wrist placement, right-hand wrist placement, left ankles placement, and right ankles placement.

6. The health kiosk system as claimed in claim 1, wherein the computer-implemented intelligent models comprises at least one of a: self-aware artificial intelligence model, large language model, supervised artificial intelligence and machine learning model, unsupervised artificial intelligence and machine learning model, deep learning model, simple and multimodal analytics including at least one of: regression models, Gaussian Mixture Models (GMMs), and K-Nearest Neighbors Algorithm models.

7. The health kiosk system as claimed in claim 1, wherein the survey generation subsystem is configured to dynamically generate health-related queries based on the user's historical health data and ongoing health conditions.

8. The health kiosk system as claimed in claim 1, wherein the first data comprises at least one of: biographical information, health history data, user preferences, survey responses, choice selections for the consent data, and medical records.

9. The health kiosk system as claimed in claim 1, wherein the one or more parameters comprises at least one of a: blood pressure, body composition, body temperature, electrocardiogram (ECG) data, weight and height measurements, optical acuities data, electroencephalogram, Lung function test, urine test, oxygen saturation, Body Mass Index (BMI), blood glucose, and lipid profile.

10. The health kiosk system as claimed in claim 1, wherein the remote assistant subsystem configured with at least one of: audio conferencing capabilities and video conferencing capabilities for activating at least one of a: camera, and microphone embedded in the health kiosk unit.

11. A method managing health of one or more users based on dynamic extraction of one or more parameters, comprising:
generating, by one or more embedded sensors, sensor data;
generating, by a plurality of electrocardiogram (ECG) leads, cardiac electrical activity data of the one or more users associated with a user profile;
determining, by a decision-making subsystem configured with computer-implemented intelligent models, a type of data to be collected from the one or more users based on the user profile;
generating, by a survey generation subsystem, one or more queries for obtaining first data based on the determined type of data to be collected from the one or more user;
receiving, by a user interaction subsystem, the first data from the one or more users associated with the user profile through a user interface to initiate personalised health-related interactions within the health kiosk system;
processing, by a data processing subsystem, at least one of the: sensor data, cardiac electrical activity data and the first data for determining the one or more parameters of the one or more users associated with the user profile;
connecting, by a remote assistant subsystem, the one or more users with a healthcare professional based on the one or more parameters for virtual assistance; and
allowing, by a remote test triggering subsystem, the healthcare professional to trigger a medical screening test on the one or more users interacted with the health kiosk unit for managing the health of the one or more users based on dynamic extraction of the one or more parameters.

12. The method as claimed in claim 11, comprises:
obtaining, by a consent data obtaining subsystem, consent data from the one or more users to recommend optimised preferences,
wherein the consent data enable the one or more users to manage and optimise data-sharing preferences for at least one of: telemedicine, research, and analysis.
